(19) United States
(12) Patent Application Publication
(10) Pub. No.: US 2023/0386641 A1
(43) Pub. Date: Nov. 30, 2023

(54) SYSTEMS AND METHODS FOR IDENTIFYING BACTERIAL POTENTIAL AND IMPACT VALUE OF INGESTIBLE PRODUCTS

(71) Applicant: Audric M. LEBOVITZ, Falls Church, VA (US)

(72) Inventor: Audric M. LEBOVITZ, Falls Church, VA (US)

(21) Appl. No.: 18/201,894

(22) Filed: May 25, 2023

Related U.S. Application Data
(60) Provisional application No. 63/346,138, filed on May 26, 2022.

(51) Int. Cl. G16H 20/60; G16H 50/30

(52) U.S. Cl. CPC G16H 20/60; G16H 50/30

(57) ABSTRACT

A method of promoting an activity, health-goal, disease amelioration-state of a mammal by consuming an ingestible product is provided. The method comprises determining an impact value or bacterial potential of an ingestible product; and consuming the ingestible product which has a positive impact value or bacterial potential on the mammal's gut microbiome for the activity, health-goal, or disease amelioration state. The impact value comprises identifying the effect of the compounds which comprise the ingestible product on one or more physiological pathways of the microbiome and/or the mammal's physiology. The activity can be playing a recreational sport, dieting, sleeping, or studying.

### DESCRIPTION OF THE PROCESS

Yorgut is a tool that provides a gateway between food and food-related endeavors. A food-related endeavor is essentially anything that relates to food, including identifying how foods affect health, disease, activity, and performance; identifying the content of a food, of a menu, of a prepared food, and of other ingestible items and how it impacts health and disease; identifying foods affecting a microbiome, physiology, etc., of a mammal ingesting the food; identifying restaurants that provide meals that meet any of the identified foods; identifying where food, and nutritional and supplements can be purchased; identifying food and diet social networks; etc.

One aspect of Yorgut is a functional food tool that identifies the impact of a food on the microbiome and on the physiology of the mammalian organism ingesting the food. The impact can be hierarchical, where the food impacts the microbiome which in turn impacts the mammal's physiology or vice-versa, or in parallel, where the food's impact on the microbiome and the mammal's physiology is each determined independently.

We refer to the functional food tool which identifies how a food impacts the organism's microbiome as the microbiome functional tool or MFT. The MFT is based on a unique formula that provides an impact value of a food on the mammalian organism. MFT is a tool that enables the mammalian organism to choose the foods, supplements, drugs, etc., which promote the health of the organism without deleterious effects, as well as determine which to avoid. In one aspect, MFT provides a “bacterial potential” for each food and food component to indicate the food's effect on a bacteria's metabolic pathways.

MFT uses the physiology of the microbiome organisms, such as bacteria, viruses, protista, etc., and host to derive microorganism potential, such as bacterial potentials and impact values on the mammal ingesting the food. The impact values are values which quantitate the effect of a food on a desired outcome. For example, if the user wants to select a healthy food, a food for a specific physical activity or mental state; a food to promote weight loss, a food to avoid exacerbating one or more symptoms of a disease, improve disease outcomes, etc., the MFT tool uses the microbiome composition to derive bacterial, etc., potentials and impact values to achieve the desired outcome.

“Food” is used generally to mean any product that is ingested by an organism, including conventional foods, such as fruits, vegetables, snacks, prepared foods, meats, fish, candy, grains, beans etc. While this disclosure is generally written in terms of “foods,” it is applicable to any product that is consumed or administered to a mammalian organism, including food and nutritional supplements, drugs, etc. For example, the system described in this disclosure can used to determine the impact of a drug on the microbiome.

The organism can be a human, a mammal, a pet, dog, cat, rabbit, hamster, rat, bird, fish, reptile, a farm animal, cow, pig, sheep, chicken, turkey, horse, ferret, wild animals, etc.

There are many different ways in which the MFT tool can be designed to derive impact values. While the formula used in the tool is ultimately derived from a natural behavior/physiological attribute/metabolic attribute of a bacterial component of the microbiome and host organism physiology, it only uses this information as a building block to make determinations about the outcome using formula. The impact value is not a natural value, but rather is an aggregate of information derived from predicted, empirical, and/or learned data.

The impact value can be determined based on each compound's effect on the microbiome's pathway, all at once or individually, to determine the bacteria potential of each compound to interact with the pathway. A positive impact value or bacteria potential is a value that promotes or facilitates the production of bacterial compound, etc., that, in turns promotes or facilitates a mammalian pathway or mammalian activity. A negative impact value or bacterial potential inhibits or impedes the pathways and/or activities. While this disclosure is written in terms of bacteria, the impact of any microorganism residing in the gut can be determined and used in this analysis.

The impact value is integrated into practical implementations of Yorgut. For example, if a user desires to determine the effect of a food on the gut microbiome, the impact value provides guidance to the user on whether to consume the food to achieve the desired outcome. In other words, the process described here is not simply conceptual and mental, but provides actual direction on what foods to eat and what foods not to eat. A person desiring to enhance its metabolism for dieting purposes, for energy to play basketball, for an afternoon of intensive study, or to treat a specific disease condition (ulcerative colitis, inflammatory bowel syndrome, flatulence, constipation and other intestinal conditions; allergy), will use MFT to determine what food will benefit/detriment these activities/conditions and then the person will actually consume them or actively avoid them. The paradigm is similar to a patient being diagnosed and treated with a drug to achieve a desired therapeutic outcome. Yorgut provides a tool to identify what food, supplement, etc., is beneficial to organism. For example, MFT has been used to determine that grilled chicken promotes staphylococcus in the gut, a bacteria that can be deleterious to the gut, advising the mammal to avoid grilling chicken if susceptible to staphylococcus.

Any microbiome can be used in combination with the MFT. The gut microbiome comprises over 10¹¹ to 10¹² bacteria per milliliter in the colon and more than 1000 different species. A large number of studies have shown the importance of the gut microbiome.

Yorgut can use a mammalian standardized microbiome, e.g., from a human, dog, cat, etc., which can be further selected based on the subject's genotype, gender, diet, phenotype, age, activity level, mental state, weight, height, etc. A personalized microbiome based on the sampling of the individual's microbiome can also be used, e.g., from feces, oral bacteria, skin bacteria, etc.

There are numerous practical implementations of Yorgut.

In one implementation, a user is presented with a graphical user interface which provides a menu of selectable items, such as: MFT; links to food activities, such as social communities/networks, product sales, restaurants, menus, etc. Each item on the menu can be tailored individually, or at once, to the individual's lifestyle. For example, a profile of a user who eats organic food and is dieting, when selecting the“menu” link, will obtain results of interest to a person who eats organic foods and is dieting.

There are also numerous practical implementations of MFT. Initially, we discuss two major ways in which MFT works:

First, the user has the option of selecting a food and an outcome. MFT displays the impact value or bacterial potential (e.g., as a number, color, symbol, etc) which provides the direction to the user on whether to consume the food.

Second, the user has the option of selecting an outcome and MFT displays the bacterial potential or impact value for foods which promote or impede the outcome. The food is displayed to the user along with a bacterial potential/impact value for it, providing the instruction to the user on whether to consume the food to achieve the desired outcome.

As an example, a user intends to spend an intensive day studying and seeks to identify the foods that positively impact concentration, focus, etc., that promote studying. The user can input a meal into the MFT interface, select “study” as the outcome, and receive an output that informs the user whether the prospective meal is compatible with intensive studying. Yorgut does not end at the display of the food. Yorgut provides links to services/suppliers providing the food (such as restaurants and maps to them), social networks with the same goals, and generally, anything of relevance to a person who eats food. The links, as described above, can be provided as a menu in a graphical user interface.

The MFT interface provides the components or fields that enable the user to interact with it. For example, the interface can comprise the following components: menu to select the desired outcome; entry field for entering the food; output field bacterial showing impact values and bacterial potentials; links to services, products, etc., relevant to the desired outcome. As indicated above, the interactive menu and search results can be based on the user's selections (studying, sports, dieting), profile, and MFT output. For example, if the user is looking at the effects of ice cream on health, on an activity (dieting), etc., the menu would provide a selectable link to ice cream stores and suppliers, as well as the impact values of different ice creams.

Yorgut also has a media sharing module, in which users can upload, download, and share media between users of a social network.

The MFT is one aspect of Yorgut which makes determinations about the impact of a food, drug, supplement, etc. on the selected activity based on the microbiome, particularly the gut microbiome. Any aspect of the microbiome can be used to determine an impact value.

The databases used by Yorgut comprise:
Food database (content/composition of food products);
Product database (content/composition of supplements, drugs, other ingestible non-food products);
Gut microbiome database (microbiome bacteria indexed by species, gender, age, activity level, etc);
Metabolic and physiological pathway database (bacteria, human);
Interaction database (interactions between host, bacteria)

MFT can be used to identify healthy and unhealthy foods. For example, if the user wants to select a food for a healthy colon, MFT can be used to identify foods that serve this purpose. Butyrate is one example because certain diseases, such as colon cancer, ulcerative colitis, and type II diabetes have been linked to perturbations in butyrate. MFT enables a user to make a meal using foods that promote butyrate, and avoiding foods that are deleterious to butyrate production.

Butyrate pathways and the bacteria which possess them are known. For example: Firmicutes, Actinobacteria, Bacteroidetes, Fusobacteria, Proteobacteria, Spirochaetes, and Thermotoga are known to possess butyrate pathways. See Vital M, Howe A C, Tiedje J M. 2014. Revealing the bacterial butyrate synthesis pathways by analyzing (meta)genomic data. mBio 5(2):e00889-14. doi:10.1128/mBio.00889-14. Substrates for these pathways include amino acids, resistant dietary carbohydrates, lactate, succinate. Yorgut takes this information and uses its unique approach to determine impact values that are subsequently employed to produce a meal that the user actually consumes.

1. A method of promoting an activity, health-goal, disease amelioration-state of a mammal by consuming an ingestible product that promotes the activity, comprising:
determining an impact value or bacterial potential of an ingestible product; and
consuming the ingestible product which has a positive impact value or bacterial potential on the mammal's gut microbiome for the activity, health-goal, or disease amelioration state.

2. The method of claim 1, where determining the impact value or bacterial potential comprises identifying the effect of the compounds which comprise the ingestible product on one or more physiological pathways of the microbiome.

3. The method of claim 1, where determining the impact value or bacterial potential comprises identifying the effect of the microbiome physiological pathways on the mammal's physiology.

4. The method of claim 1, where determining the impact value comprises determining the bacteria potential of each compound.

5. The method of claim 1, where the activity is playing a recreational sport, dieting, sleeping, or studying.

6. A method comprising:
(a) a user selecting an activity, health-goal, or disease-state of a mammal;
(b) identifying the physiological pathways in the mammal which facilitates the activity, health-goal, or disease-state;
(c) determining the pathways in the microbiome of the mammal which promote the mammalian pathways associated with the selected activity, health-goal, or disease-state;
(d) identifying ingestible product components which promote or inhibit the determined microbiome pathways;
(e) determining a food which contains the identified ingestible product components;
(f) consuming the determined food which promotes the determined microbiome pathways and not consuming the determined food which inhibits the determined microbiome pathway;
where determining the pathways comprising determining an impact score or bacterial potential.

7. A method comprising:
(a) determining the dietary and chemical components of an ingestible product;
(b) determining the microbiome of the mammal;
(c) identifying the effect of the components and/or ingestible product on the microbiome;
(d) determining a first impact score based on the effect of the components and/or ingestible product on the microbiome;
(e) determining a second impact score based on the effect of one or more microbiome compounds produced by the microbiome of the mammal;
(f) determining the effect on the mammal based on the first and/or second impact score;
(g) consuming the ingestible product by the mammal, where the impact score shows that the food has a desired impact on an activity of a mammal.

8. The method of claim 7, wherein the mammalian activity is sleep, exercise, diet, sports, health-promotion, or focus.

9. A system for carrying out the method of claim 1, comprising:
a menu having selectable links to a food, a menu, or a restaurant, where the food is identified from the method of claim 1.
